(19) United States
(12) Patent Application Publication
(10) Pub. No.: US 2023/0190242 A1
(43) Pub. Date: Jun. 22, 2023

(54) VIRAL SAMPLE COLLECTION

(71) Applicant: THOMAS JEFFERSON UNIVERSITY, Philadelphia, PA (US)

(72) Inventor: William Martin Keane, Gladwyne, PA (US)

(21) Appl. No.: 17/925,975

(22) PCT Filed: May 17, 2021
(86) PCT No.: PCT/US2021/032698

§ 371 Date: Nov. 17, 2022

Related U.S. Application Data
(60) Provisional application No. 63/075,571, filed on Sep. 8, 2020.
(60) Provisional application No. 63/026,539, filed on May 18, 2020.

(51) Int. Cl. A61B 10/00; C12Q 1/70

(52) U.S. Cl. CPC A61B 10/0051; C12Q 1/70; A61B 2010/0006

(57) ABSTRACT

The present invention includes a method of obtaining a biological sample from the nose, nasal cavities, nasal pharynx, oral mucosa, or saliva of a subject to be used for detection of a virus (e.g., a SARS-Co V-2 virus), and kits for performing the method.

### CROSS-REFERENCE TO RELATED APPLICATIONS

This application claims the benefit of priority under 35 U.S.C. § 119(e) to U.S. Provisional Patent Application Nos. 63/026,539, filed May 18, 2020, and 63/075,571, filed Sep. 8, 2020, all of which applications are incorporated herein by reference in their entireties.

### BACKGROUND OF THE INVENTION

COVID-19 is an infectious disease caused by the coronavirus SARS-CoV-2. The virus is believed to spread mainly from person to person. People with COVID-19 have had a wide range of symptoms reported, ranging from mild symptoms to severe illness. As of May 10, 2020, according to the Centers for Disease Control and Prevention (CDC), 1,300,696 cases and 78,771 deaths due to COVID-19 have been reported in the United States.

A need exists for improved methods of testing persons for viruses such as SARS-CoV-2. In particular, a need exists for improved methods for collecting samples of viruses that pass through nasal cavity, such as SARS-CoV-2, and enhancing the quality of the samples used for testing. The present invention addresses and satisfies this need.

### SUMMARY OF THE DISCLOSURE

In one aspect, the present disclosure provides a method of obtaining a biological sample from the nose, nasal cavities, nasal pharynx, oral mucosa, or saliva of a subject for detection of a virus, the method comprising: (a) inserting into the nose of the subject a saline solution comprising a dye and (b) collecting fluid from the nose or mouth of the subject. In one embodiment, in step (a), the saline solution is allowed to flow from the nose to the mouth of the subject through the nasal passage and the nasal pharynx. In one embodiment, the saline solution is allowed to wash over the contents and walls of the nasal cavities, nasal pharynx, oral mucosa, or combination thereof. In one embodiment, the saline solution is atomized. In one embodiment, the method further comprises (c) determining whether the fluid collected in step (b) contains a sufficient amount of the saline solution based on the color of the fluid. In one embodiment, if the fluid collected in step (b) is determined to not contain a sufficient amount of the saline solution, steps (a)-(c) are repeated. In another embodiment, if the fluid collected in step (b) is determined to contain a sufficient amount of the saline solution, the fluid collected in step (b) is determined to be an acceptable sample for subsequent use in detection of a virus. In one embodiment, step (c) further comprises determining whether the fluid collected in step (b) contains a sufficient amount of the saline solution based on the taste of the fluid. In one embodiment, the dye is safe for ingestion. In one embodiment, the method is self-administered. In one embodiment, the subject is human. In one embodiment, the virus is a coronavirus, a rhinovirus, an influenza virus, a parainfluenza virus, or a respiratory syncytial virus. In one embodiment, the virus is SARS-CoV-2.

In another aspect, the present disclosure provides a method of detecting a virus in a biological sample from a subject, the method comprising: (a) inserting into the nose of the subject a saline solution comprising a dye; (b) collecting fluid from the nose or the mouth of the subject; and (c) detecting presence of a virus in the fluid collected in step (b). In one embodiment, in step (a), the saline solution is allowed to flow from the nose to the mouth of the subject through the nasal passage and the nasal pharynx. In one embodiment, the saline solution is allowed to wash over the contents and walls of the nasal cavities, the nasal pharynx, the oral mucosa, or a combination thereof. In one embodiment, step (c) is preceded by the step of determining whether the fluid collected in step (b) from the mouth of the subject contains a sufficient amount of the saline solution based on the color of the fluid. In one embodiment, if the fluid collected in step (b) is determined to contain an insufficient amount of the saline solution, steps (a) and (b) are repeated. In one embodiment, the step of determining whether the fluid collected in step (b) from the mouth of the subject contains a sufficient amount of the saline further comprises the step of determining whether the fluid collected in step (b) contains a sufficient amount of the saline solution based on the taste of the fluid. In one embodiment, the saline solution is atomized. In one embodiment, the dye is safe for ingestion. In one embodiment, the presence of the virus is detected by polymerase chain reaction (PCR). In one embodiment, the subject is human. In one embodiment, the virus is a coronavirus, a rhinovirus, an influenza virus, a parainfluenza virus, or a respiratory syncytial virus. In one embodiment, the virus is SARS-CoV-2.

In yet another aspect, the present disclosure provides a method of determining whether a subject is infected with a virus, the method comprising: (a) detecting the presence of a virus in a biological sample obtained from the subject, wherein the biological sample comprises fluid collected from the nose or the mouth of the subject after a saline solution comprising a dye is inserted into the nose of the subject and allowed to wash over the contents and walls of the nasal cavity, the nasal passage, the nasal pharynx, or combinations thereof. In one embodiment, the biological sample comprises fluid collected from the mouth of the subject after a saline solution comprising a dye is inserted into the nose of the subject and allowed to flow from the nose to the mouth of the subject through the nasal passage, the nasal pharynx, the oral mucosa, or a combination thereof. In one embodiment, the fluid collected from the mouth subject is determined to contain a sufficient amount of the saline solution based on the color of the fluid. In one embodiment, the fluid collected from the mouth subject is determined to contain a sufficient amount of the saline solution based on the taste of the fluid. In one embodiment, the saline solution is atomized. In one embodiment, the dye is safe for ingestion. In one embodiment, the presence of the virus is detected by polymerase chain reaction (PCR). In one embodiment, the subject is human. In one embodiment, the virus is a coronavirus, a rhinovirus, an influenza virus, a parainfluenza virus, or a respiratory syncytial virus. In one embodiment, the virus is SARS-CoV-2.

In yet another aspect, the present disclosure provides a kit for performing a method disclosed herein, wherein the kit comprises a saline solution comprising a dye.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIG. 1 provides a view of the nasal interior wherein the major components are labelled.

FIG. 2 depicts the distance that an atomized plume of saline solution comprising a dye can travel after ejection from an atomizer or squeeze/squirt bottle (about 15 inches).

FIG. 3 provides an exemplary kit for the collection of biological samples using the disclosed method.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

The term “antibody” or “Ab” as used herein, refers to a protein, or polypeptide sequence derived from an immunoglobulin molecule, which specifically binds to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv) and humanized antibodies (Harlow et al., 1998, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). An antibody may be derived from natural sources or from recombinant sources. Antibodies are typically tetramers of immunoglobulin molecules.

The term “ameliorating” or “treating” means that the clinical signs and/or the symptoms associated with a disease are lessened as a result of the actions performed. The signs or symptoms to be monitored will be well known to the skilled clinician.

As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The term “sample” or “biological sample” refers to a sample obtained from an organism or from components (e.g., cells) of an organism. The sample may be any biological tissue or fluid. The sample can be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes. In particular embodiments, the biological sample is fluid collected from a subject's mouth following nasal irrigation. In some embodiments, the biological sample is tested for the presence for SARS-CoV-2 virus.

As used herein, the terms “control,” or “reference” are used interchangeably and refer to a value that is used as a standard of comparison.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “specifically binds”, “selectively binds” or “binding specificity” refers to the ability of the humanized antibodies or binding compounds of the invention to bind to a target epitope with a greater affinity than that which results when bound to a non-target epitope. In certain embodiments, specific binding refers to binding to a target with an affinity that is at least 10, 50, 100, 250, 500, or 1000 times greater than the affinity for a non-target epitope.

The term “expression” as used herein is defined as the transcription and/or translation of a particular nucleotide sequence driven by its promoter.

As used herein, the term “expression cassette” means a nucleic acid sequence capable of directing the transcription and/or translation of a heterologous coding sequence. In some embodiments, the expression cassette comprises a promoter sequence operably linked to a sequence encoding a heterologous protein. In some embodiments, the expression cassette further comprises at least one regulatory sequence operably linked to the sequence encoding the heterologous protein.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “RNA” as used herein is defined as ribonucleic acid.

“Transform”, “transforming”, and “transformation” is used herein to refer to a process of introducing an isolated nucleic acid into the interior of an organism.

The term “treatment” as used within the context of the present invention is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

The term “equivalent,” when used in reference to nucleotide sequences, is understood to refer to nucleotide sequences encoding functionally equivalent polypeptides. Equivalent nucleotide sequences will include sequences that differ by one or more nucleotide substitutions, additions- or deletions, such as allelic variants; and will, therefore, include sequences that differ from the nucleotide sequence of the nucleic acids described herein due to the degeneracy of the genetic code.

The term “isolated” as used herein with respect to nucleic acids, such as DNA or RNA, refers to molecules separated from other DNAs or RNAs, respectively that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments, which are not naturally occurring as fragments and would not be found in the natural state. The term “isolated” is also used herein to refer to polypeptides, which are isolated from other cellular proteins and is meant to encompass both purified and recombinant polypeptides. An “isolated cell” or “isolated population of cells” is a cell or population of cells that is not present in its natural environment.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids. As used herein, nucleic acids include but are not limited to, all nucleic acid sequences which are obtained by any means available in the art, including, without limitation, recombinant means, i.e., the cloning of nucleic acid sequences from a recombinant library or a viral genome, using ordinary cloning technology and PCR, and the like, and by synthetic means.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, the term “pharmaceutical composition” refers to a mixture of at least one compound useful within the invention with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the term “effective amount” or “therapeutically effective amount” means the amount of a therapeutic which is required to prevent the particular disease condition, or which reduces the severity of and/or ameliorates the disease condition or at least one symptom thereof or condition associated therewith.

A “subject” or “patient,” as used therein, may be a human or non-human mammal. Non-human mammals include, for example, livestock and pets, such as ovine, bovine, porcine, canine, feline and murine mammals. Preferably, the subject is human. In some embodiments, the subject is a domestic pet or livestock. In some embodiments, the subject is suspected to have COVID-19. In some embodiments, the subject has COVID-19.

“Titers” are numerical measures of the concentration of a virus or viral vector compared to a reference sample, where the concentration is determined either by the activity of the virus, or by measuring the number of viruses in a unit volume of buffer. The titer of viral stocks are determined, e.g., by measuring the infectivity of a solution or solutions (typically serial dilutions) of the viruses, e.g., on HeLa cells using the soft agar method (see, Graham & Van der Eb (1973) Virology 52:456-467) or by monitoring resistance conferred to cells, e.g., G418 resistance encoded by the virus or vector, or by quantitating the viruses by UV spectrophotometry (see, Chardonnet & Dales (1970) Virology 40:462-477).

“Vaccination” refers to the process of inoculating a subject with an antigen to elicit an immune response in the subject, that helps to prevent or treat the disease or disorder the antigen is connected with. The term “immunization” is used interchangeably herein with vaccination.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In the present disclosure, the term “vector” includes an autonomously replicating virus.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

Described herein is a novel methodology on collecting viral samples from the upper airway which can be self-administered and does not require the use of a probe. The method of collection comprises irrigation of the nasal cavities with a colored saline solution (e.g., a saline solution comprising a dye) for enhancement of collection of viral samples from a patient wherein such saline solution is inserted into the nose and allowed to wash through the nasal cavities. In some embodiments, the solution is passed through the nasal pharynx and then collected from the mouth of the patient. In some embodiments wherein the solution is collected from the mouth of the patient, the solution washes the oral mucosa and contains saliva from the mouth of the patient. in some embodiments, the solution is collected from the nose of the patient. In some embodiments, the saline solution is atomized. This technique allows samples to be obtained from those parts of the nose, nasal cavities, and nasal pharynx which contains the bulk of the viral particles. In embodiments wherein the sample is collected from the mouth of the patient, the sample will further contain viral particles from the oral mucosa and/or saliva of the patient. The virus can be any virus that passes through the nasal cavity, e.g., a SARS virus such as SARS-CoV-2, or an influenza virus.

The use of a dye in the aforementioned saline solution allows one to confirm adequate nasal irrigation with effective sampling of the nose and nasal pharynx to allow one to visibly confirm that the fluid collected from the mouth or nose of the patient in fact contains a. sufficient amount of the nasal saline irrigation solution. Furthermore the use of the saline allows the patient to taste solution assuring passage from the nose to the mouth. A key element of the test is that it can be self-administered at any site of service, including one's home.

Also described herein is a kit comprising a colored saline solution that will be used to flush particles, e.g., viral particles from the nose and nasal pharynx. The saline solution can be colored with a vegetable dye or other dye safe for ingestion. Such particles can then be pulled into the mouth and spit and/or blown into a collecting cup containing different types of viral reagents. The sample solution will then be sealed and packaged in a way to prevent contamination.

### Method of Collecting Sample

In one aspect, a method of obtaining a biological sample from the nose of a subject is provided. The method uses nasal irrigation to collect biological samples to be used for testing for the presence of a virus, e.g., SARS-CoV-2 virus.

Current methods used to collect samples for detection of SARS-CoV-2 or diagnosis of COVID-19 rely on a nasal swab passed along the floor of the nose to try to sample the mucosa of the nasopharynx. Unfortunately, a major limitation of this technique is that it likely has a high false negative rate. It is sampling only a fraction of the nasal mucosa and relies on gravity to hopefully bring viral particles down to the floor of the nose. It is tester and patient dependent. The nose is very sensitive and patients do not tolerate deep nasal probing well or probing high up in the nose to olfactory groove. Finally, it requires a trained healthcare professional to administer the swab. Unlike such methods, the methods described herein do not require a swab (e.g., a cotton swab) to be inserted into the subject's nasal cavity.

The methods described herein use a nasal cavity and/or nasopharyngeal lavage. The methods described herein are expected to be more accurate, easily self-administered, and more cost effective than current methods that rely on use of a nasal swab. In some embodiments, the methods comprises a self-administered nasal cavity and nasopharyngeal lavage using dyed saline administered with an atomizer. The lavage will bathe the entirety of the nasal mucosa, including the mucosa of the olfactory groove. The lavaged saline can then be allowed to pass from the back of the nose into the mouth, wherein it will bathe the oral mucosa and mix with the patient's saliva. The saline can then be expectorated into a specimen collection container that has stabilizing reagent. In some other embodiments, the lavaged saline is blown out and collected from the nose.

In one embodiment, a method of obtaining a biological sample from one or more of: the nose, nasal cavities, nasal pharynx, oral mucosa, or saliva, or any combination thereof, of a subject, is provided. The sample collected from the nose, nasal cavities, nasal pharynx, oral mucosa, or saliva, or any combination thereof, can be assayed for a virus (e.g., a SARS-CoV-2 virus) to determine whether the virus is present or absent in the sample.

In one embodiment, the method includes the steps of (a) inserting into the nose of a subject a saline solution comprising a dye and (b) collecting fluid from the nose or the mouth of the subject.

In some embodiments, in step (a), the saline solution is allowed to pass through and wash over the contents and walls of the nasal cavities and passageway. In some embodiments, in step (a), the saline solution is further allowed to pass through and wash the nasal pharynx. In certain embodiments, in step (a), the saline solution is allowed to flow from the nose to the mouth of the subject through the nasal passage and nasal pharynx. In some embodiments, as the saline solution flows from the nose to the mouth of the subject, the solution washes the oral mucosa and mixes with saliva in the subject's mouth.

In some embodiments, in step (a), the saline solution is atomized. The saline solution can be atomized using any method. In one embodiment, the saline solution is atomized using an atomizer. In some embodiments, the atomizer is of the appropriate size or length and/or has a stopper on it to prevent insertion of the atomizer too far up the nose of the subject. In some embodiments, the atomizer creates a large, but gentle, plume of saline that adequately bathes the surface of the entire nasal cavity, including the olfactory groove up high and all of the surfaces of the nasal turbinates. This can increase the chance of collecting viral particles if present and can decreases the risk of a false negative result. In another embodiment, the saline solution is atomized as it is ejected from a squeeze or squirt bottle.

In some embodiments, the method further includes the step of (c) determining whether the fluid collected from the subject's mouth contains a sufficient amount of the saline solution based on the color of the fluid. Use of a dye in the saline solution allows confirmation that the fluid contains a sufficient amount of the saline solution by visual inspection of the fluid, indicating that the nasal irrigation was successful and the fluid collected is acceptable for use as a sample to be tested for a virus, e.g., SARS-CoV-2. In embodiments wherein, in step (b), fluid is collected from the mouth of the subject, insufficient color (e.g., no color or no dye) could indicate inadequate irrigation and further indicate that any viral particles in the subject's nose or nasopharynx were not properly flushed or collected. Thus, in some embodiments, if insufficient color or dye is detected in the fluid collected, nasal irrigation with the saline solution comprising the dye (e.g., step (a)) and/or collection of fluid from the subject's mouth (e.g., step (b)) is repeated. The steps may be repeated until the fluid sample collected from the subject's mouth shows sufficient color, indicating adequate irrigation.

In some embodiments, in step (b), the fluid, e.g., fluid that has washed the contents and walls of the nasal cavities and passageways is collected from the nose of the subject. For example, the fluid can be blown from the nose.

Use of a saline solution also allows the subject to taste the solution when the solution is in the subject's mouth. Thus, the taste of the fluid collected from the subject's mouth can also be used as in indicator of adequate or successful irrigation. In some embodiments, the method further comprises the step of determining whether the fluid collected contains a sufficient amount of the saline solution based on the taste of the fluid.

The saline solution can be any saline solution, e.g., commercially available saline solutions used in nasal irrigation. In some embodiments, the saline solution comprises a salt (e.g., NaCl) dissolved in water (e.g., distilled water). In some embodiments, the saline solution has a salinity between 0.9% to 3%. In some embodiments, the saline solution is sterile.

The dye used to color the saline solution can be any dye. Preferably, the dye is non-toxic and safe for ingestion. In some embodiments, the dye is a vegetable dye. In some embodiments, the dye does not interfere with further processing of the fluid sample and/or assays to be performed on the fluid sample. When inspecting the color of the fluid collected from the subject's mouth to determine whether the irrigation was successful or to determine if the fluid is acceptable for subsequent use in testing for a virus (e.g., SARS-CoV-2), the color of the fluid may be compared to a reference. The reference may be a control, e.g., the color of a saline solution without the dye that is passed through the subject's nose, nasal cavities and nasal pharynx. The reference can also be a pre-determined threshold value, e.g., a pre-determined color intensity or shade. In some embodiments, the fluid collected is determined to contain an a sufficient amount of saline solution if the color of the solution is about the same or increased relative to a reference. In some embodiments, the fluid sample collected is determined to contain an insufficient amount of saline solution if the color of the dye is not detected (e.g., not seen visually) in the fluid sample.

In some embodiments, the method is self-administered. In some embodiments, the nasal irrigation with the colored solution and/or fluid collection steps are performed by the same subject whose biological sample is collected. In some embodiments, the determination of whether the fluid collected contains a sufficient amount of saline solution (e.g., by visual inspection of the color of the fluid and/or taste of the fluid) is performed by the same subject whose biological sample is collected.

In some embodiments, the subject whose biological sample is collected has COVID-19. In some embodiments, the subject whose biological sample is collected is suspected of having COVID-19.

Fluid samples that have been determined to contain a sufficient amount of the saline solution are deemed acceptable or suitable samples to be used in testing for a virus, e.g., SARS-CoV-2. In some embodiments, the fluid samples are placed in a container that contains reagents, e.g, reagents for preserving or stabilizing the sample, for further processing of the sample, and/or for detection of virus in the sample. In one embodiment, the fluid samples are placed in a container that contains a stabilizing reagent. The stabilizing reagent can be any stabilizing reagent. In some embodiments, the stabilizing reagent is a reagent known to stabilize RNA samples, such as, but are not limited to, liquid Amies, phosphate buffered saline (PBS), EDTA, Tris EDTA (TE) buffer, and combinations thereof. In some embodiments, the fluid samples are sealed and/or packaged in a container in a manner that prevents contamination of the sample. In some embodiments, the sample or specimen collection container has a one-way valve to prevent the subject from drinking the stabilizing reagent.

The fluid sample can then be tested for presence of a virus in the sample. In some embodiments, the virus is a virus that passes through the nasal cavity. In some embodiments, the virus is a respiratory virus. In some other embodiments, the virus is a coronavirus, a rhinovirus, an influenza virus (e.g., influenza A virus, influenza B virus), a parainfluenza virus, or a respiratory syncytial virus (RSV). In some embodiments, the coronavirus is a SARS virus. In some embodiments, the virus is SARS-CoV-2.

In another aspect, a method of detecting a virus in a biological sample from a subject is provided. The method includes the steps of (a) inserting into the nose of the subject a saline solution comprising a dye; (b) collecting fluid from the nose or mouth of the subject; and (c) detecting presence of a virus in the fluid collected in step (b). In some embodiments, in step (a), the saline solution is allowed to pass through and wash over the contents and walls of the nasal cavities and passageway. In some embodiments, in step (a), the saline solution is further allowed to pass through and wash the nasal pharynx. In certain embodiments, in step (a), the saline solution is allowed to flow from the nose to the mouth of the subject through the nasal passage and nasal pharynx. In some embodiments, as the saline solution flows from the nose to the mouth of the subject, the solution washes the oral mucosa and mixes with saliva in the subject's mouth. In some embodiments, in step (a), the saline solution is atomized. The saline solution can be atomized using any method. In one embodiment, the saline solution is atomized using an atomizer. In another embodiment, the saline solution is atomized as it is ejected from a squeeze or squirt bottle. In some embodiments, the virus is SARS-CoV-2.

In some embodiments, step (c) is preceded by the step of determining whether the fluid collected in step (b) from the mouth of the subject contains a sufficient amount of the saline solution based on the color of the fluid. In one embodiment, the step of detecting presence of a virus in the fluid collected in step (b) does not occur if the sample is determined to contain an insufficient amount of saline solution based on the color of the fluid. In some embodiments, if the fluid collected in step (b) is determined to contain an insufficient amount of the saline solution, steps (a) and (b) are repeated.

In another aspect, a method of determining whether a subject is infected with a virus is provided. In yet another aspect, a method of diagnosing a subject with a viral infection or viral disease is provided. The methods include detecting the presence of a virus in a biological sample obtained from the subject. In some embodiments, the wherein the biological sample comprises fluid collected from the mouth of the subject after a saline solution comprising a dye is inserted into the nose of the subject and allowed to flow from the nose to the mouth of the subject through the nasal passage and the nasal pharynx. In some embodiments, as the fluid is allowed to flow from the nose to the mouth of the subject, the fluid washes the oral mucosa and mixes with the subject's saliva. In another embodiment, the biological sample comprises fluid collected from the nose of the subject after a saline solution comprising a dye is inserted into the nose of the subject and allowed to contact and coat the nasal cavity and nasal passages. In some embodiments, atomized saline solution is inserted into the nose of the subject. In some embodiments, the atomized saline solution is ejected from an atomizer or a squirt/squeeze bottle into the nose of the subject. In some embodiments, the saline solution is allowed to pass through and wash over the contents and walls of the nasal cavities and passageway, and to pass through and wash the nasal pharynx. In some embodiments, the biological samples used in testing or detection of a virus are samples determined to be suitable for use in the testing, e.g., samples that were determined to contain sufficient saline solution, as indicated by the color and/or taste of the fluid sample. In some embodiments, the virus is SARS-CoV-2. In some embodiments, the viral infection or viral disease is COVID-19.

In some embodiments, the step of testing for the presence of a virus or the step of detecting the presence of a virus in the sample is performed at a testing facility.

Detection of a virus in the biological sample can be carried out by standard methods. In particular embodiments, the virus can be detected by any means of detection of polypeptides (e.g., viral polypeptides), or detection of nucleic acid (e.g., viral nucleic acid). For example, the polypeptide can be detected using any of antibody detection methods (e.g., immunofluorescent (IF) methods, flow cytometry, and fluorescence activated cell sorting (FACS)), antigen retrieval and/or microarray detection methods. A reagent that specifically binds to a viral polypeptide, e.g., an antibody, an antibody derivative, and an antibody fragment, can be used. A reagent that specifically binds to a viral nucleic acid, e.g., a primer or probe, can be used in a PCR or other nucleic acid hybridization technology (e.g., microarrays). Other detection techniques that can be used include, e.g., capture assays (e.g., ELISA), mass spectrometry (e.g., LCMS/MS), and/or polymerase chain reaction (e.g., RT-PCR).

In some embodiments, a polymerase chain reaction (PCR) is performed on a biological sample to detect viral nucleic acid. In some embodiments, the PCR is quantitative PCR (qPCR). In some embodiments, the virus to be detected in the biological sample is a virus that passes through the nasal cavity. In some embodiments, the virus is a respiratory virus. In some other embodiments, the virus is a coronavirus, a rhinovirus, an influenza virus (e.g., influenza A virus, influenza B virus), a parainfluenza virus, or a respiratory syncytial virus (RSV). In some embodiments, the coronavirus is a SARS virus. In some embodiments, the virus is SARS-CoV-2. In some embodiments, a polymerase chain reaction (PCR) is performed on the biological sample to detect SARS-CoV-2 nucleic acid.

### Kits

In some aspects, a kit is provided for collection of a biological sample from a subject to be used for testing for a virus, e.g., SARS-CoV-2. In some embodiments, the subject has or is suspected of having a respiratory infection or disease, e.g., a cold, a flu, or COVID-19. In some embodiments, the subject has or is suspected of having COVID-19. In some embodiments, the kit comprises a saline solution comprising a dye, e.g., a vegetable dye or other dye safe for ingestion. In some embodiments, the kit comprises one or more components of the saline solution and/or dye, for example, the kit may comprise a dye packaged separately from the saline solution, with instructions for adding/mixing with the saline solution, or the kit may comprise components of the saline solution (e.g., salts, buffers) with instructions for preparing the saline solution from the components.

In some other embodiments, the kit comprises a container (e.g., a syringe or a squirt/squeeze bottle) for dispensing the saline solution into the nose of a subject. In one embodiment, the kit comprises a 2 cc or 3 cc syringe or a similarly sized squirt/squeeze bottle. The container may be adapted to allow the saline solution to be delivered into the subject's nose (e.g., nasal passage, nasal cavity, and nasal pharynx) with sufficient pressure. The kit may comprise other components to aid in the delivery of the saline solution or to aid in generating sufficient pressure during delivery of the saline solution to the subject's nose and nasal cavities. In some embodiments, the kit comprises a syringe, e.g. a 3 cc syringe, with an atomizer attached.

In some other embodiments, the kit comprises an atomizer. In one embodiment, the atomizer can be connected to an end of the syringe or squirt/squeeze bottle such that the saline solution can be ejected from the syringe or bottle through the atomizer.

In some other embodiments, the kit comprises a container for collecting the fluid from the subject's mouth (e.g., a cup). In some embodiments, the container contains one or more reagents, e.g., reagents used for further processing of the sample, for stabilizing or preserving the sample. In one embodiment, the container contains a stabilizing reagent. In some other embodiments, the container is adapted to be sealed, e.g., to preserve and protect the sample in the container from contamination or to prevent the sample from leaking out of the container. In some embodiments, the container is suitable for mailing or sending the sample collected to a site (e.g., a testing facility) distant from the site of collection (e.g., the subject's home).

The kit can further include instructions or a label for using the kit to collect a fluid sample to be used for testing for a virus, e.g., SARS-CoV-2. For example, the kit can include instructions for performing the nasal irrigation, for collecting fluid from the subject's nose or mouth, for visually inspecting and/or tasting the colored saline solution and determining whether the fluid collected contains a sufficient amount of the saline solution, for determining if the sample is suitable to be used for testing, or for sealing, packaging, labeling, and/or sending the sample for testing.

### Treatment

In some embodiments, a subject who is determined to be infected with virus or a subject diagnosed with a viral infection or disease (e.g., cold, flu, or COVID-19) is administered with a treatment or therapy for the viral infection or disease. In some embodiments, a subject who is determined to be infected with SARS-CoV-2 or a subject diagnosed with COVID-19 is administered with a treatment or therapy for COVID-19. Treatment and/or therapy can include over-the-counter medicines, e.g., acetaminophen, to relieve symptoms; mechanical ventilation; anti-virals; and plasma therapy. Exemplary antiviral drugs include, but are not limited to, abacavir, acyclovir, adefovir, amantadine, ampligen, amprenavir, arbidol umifenovir, atazanavir, atripla, baloxavir marboxil, biktarvy, boceprevir, bulevirtide, cidofovir, cobicistat, combivir, daclatasvir, darunavir, delavirdine, descovy, didanosine, docosanol, dolutegravir, doravirine, edoxudine, efavirenz, elvitegravir, emtricitabine, enfuvirtide, entecavir, etravirine, famciclovir, fomivirsen, fosamprenavir, foscarnet, ganciclovir, ibacitabine, ibalizumab, idoxuridine, imiquimod, imunovir, indinavir, lamivudine, letermovir, lopinavir, loviride, maraviroc, methisazone, moroxydine, nelfinavir, nevirapine, nexavir, nitazoxanide, norvir, oseltamivir, penciclovir, peramivir, pleconaril, podophyllotoxin, raltegravir, remdesivir, ribavirin, rilpivirine, rimantadine, ritonavir, saquinavir, simeprevir, sofosbuvir, stavudine, taribavirin, telaprevir, telbivudine, tenofovir alafenamide, tenofovir disoproxil, tenofovir, tipranavir, trifluridine, trizivir, tromantadine, truvada, umifenovir, valaciclovir, valganciclovir, vicriviroc, vidarabine, zalcitabine, zanamivir, zidovudine, and combinations thereof. In some embodiments, the treatment and/or therapy comprises a pharmaceutically active compound believed to aid in the treatment and/or prevention of a coronavirus infection, such as SARS-CoV-2. Exemplary compounds believed to aid in the treatment and/or prevention of a coronavirus infection include, but are not limited to, remdesivir, dexamethasone, hydroxychloroquine, chloroquine, azithromycin, tocilizumab, acalabrutinib, tofacitinib, ruxolitinib, baricitnib, anakinra, canakinumab, apremilast, marillimumab, sarilumab, lopinavir, ritonavir, oseltamivir, favipiravir, umifenovir, galidesivir, colchicine, ivermectin, vitamin D, and combinations thereof. In some embodiments, a subject who is determined to be infected with a virus (e.g, SARS-CoV-2) or a subject diagnosed with a viral infection or disease (e.g., COVID-19) is quarantined or asked to self-quarantine.

The treatment may be formulated as a pharmaceutical composition. Such a pharmaceutical composition may be in a form suitable for administration to a subject (i.e. mammal), or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

Pharmaceutical compositions that are useful in the methods of the invention may be suitably developed for inhalation, oral, rectal, vaginal, parenteral, topical, transdermal, pulmonary, intranasal, buccal, ophthalmic, intrathecal, intravenous or another route of administration. The route(s) of administration is readily apparent to the skilled artisan and depends upon any number of factors including the type and severity of the disease being treated, the type and age of the veterinary or human patient being treated, and the like.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

It should be understood that the method and compositions that would be useful in the present invention are not limited to the particular formulations set forth in the examples. The following examples are put forth so as to provide those of ordinary skill in the art with a complete disclosure and description of how to make and use the methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook et al., 2012, volumes 1-4, Cold Spring Harbor Laboratory Press, NY); “Oligonucleotide Synthesis: A Practical Approach” (M. J. Gait, ed., Oxford University Press, 1984); “Culture of Animal Cells: A Manual of Basic Technique and Specialized Applications”, sixth edition (R. I. Freshney, Wiley-Blackwell, 2010); “Methods in Enzymology” (S. P. Colowick, N. O. Kaplan, et al., eds., volumes 1-650, Academic Press) “Handbook of Experimental Immunology”, fifth edition (D. M. Weir et al., Wiley, 1997); “Gene Transfer Vectors for Mammalian Cells” (J. Miller and M. P. Calos, Cold Spring Harbor Laboratory Press, NY, 1987); “Short Protocols in Molecular Biology”, fifth edition (F. M. Ausubel et al., eds., John Wiley & Sons, 2002); “Polymerase Chain Reaction: Principles, Applications and Troubleshooting”, (M. E. Babar, VDM Verlag Dr. Muller, 2011); “Current Protocols in Immunology”, J. E. Coligan et al., eds., John Wiley & Sons, 1991). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1: Nasal Irrigation of a Patient to Collect Biological Sample to be Tested for SARS-CoV-2

Nasal irrigation is performed on a patient suspected of having COVID-19 to collect a sample from the patient to be tested for SARS-CoV-2. The nasal irrigation is performed with a saline solution containing a dye safe for ingestion (e.g., a vegetable dye). To perform the nasal irrigation, a tip of the container (e.g., a syringe or squeeze bottle) containing the saline solution is placed into the patient's nostril. An amount of the solution is delivered into the patient's nose. The saline solution is then allowed to wash through the nasal passage and nasal cavities and is passed through the nasal pharynx. The patient reports tasting a salty flavor. The patient spits fluid into a cup and the fluid from the patient's mouth is visually inspected to confirm adequate nasal irrigation. The fluid shows the color of dye, confirming that the fluid contains a sufficient amount of the nasal saline irrigation solution. A sample of the fluid is then collected into a container and is sealed and packaged to prevent contamination. The sample is sent for testing for SARS-CoV-2.

### Example 2: Nasal Cavity and Nasopharyngeal Atomized Lavage for SARS-CoV-2 Specimen Collection

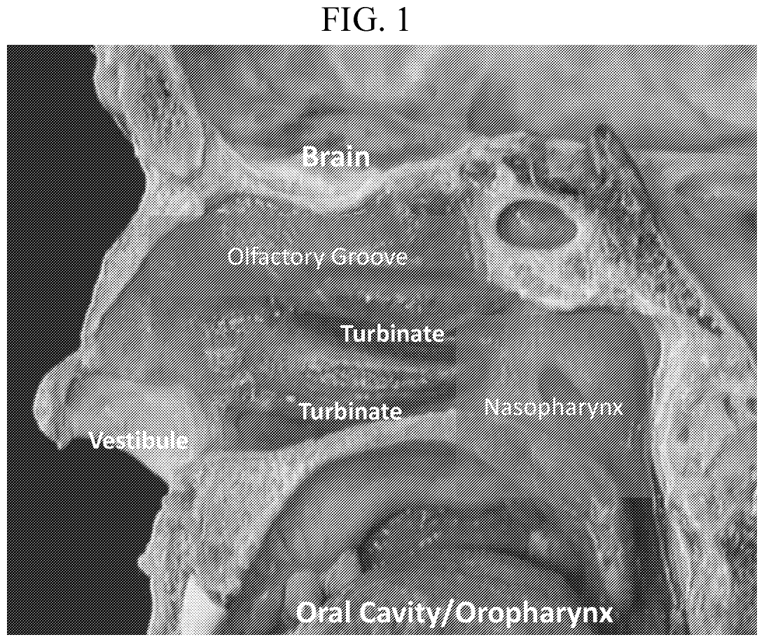

The internal nose is a complex structure that is responsible for filtering and humidifying inhaled air (FIG. 1). The lateral nasal sidewall comprises projections called turbinates that greatly increase the surface area of the nose to an estimated total surface area of 160 cm². This allows for rapid and effective humidification and filtering of air.

SARS-CoV-2, the novel coronavirus currently responsible for the COVID-19 pandemic is transferred between humans primarily via airborne transmission. As viral particles are inhaled through the nose, the filtering system of the nose filters the viral particles. However, these viral particles can then invade the all nasal mucosa, not just nasal mucosa along the floor of the nose or the nasopharynx, infecting the patient. Since the nose contains specialized smell receptors in an area called the olfactory groove and up to 70% of patients infected with SARS-CoV-2 experience smell dysfunction, it is evident that the virus particles are invading the mucosa high up in the olfactory groove.

The current test relies on a nasal swab passed along the floor of the nose to try to sample the mucosa of the nasopharynx. Because the current test is sampling only a fraction of the nasal mucosa and relies on gravity to hopefully bring viral particles down to the floor of the nose, it likely has a high false negative rate. The current test suffers from being tester and patient dependent. The nose is very sensitive and patients do not tolerate deep nasal probing well or probing high up in the nose to olfactory groove. Finally, it requires a trained healthcare professional to administer the swab.

Other proposed specimen collection techniques include anterior nasal swabbing, sputum collection, and oropharyngeal swabs. While these methods of collecting samples for detection of SARS-CoV-2 or diagnosis of COVID-19 in subjects may be more easily self-administered, once again, anterior nasal and oropharyngeal swabbing do not sample the entirety of the nasal mucosa where SARS-CoV-2 resides. The viscosity of sputum makes sputum collection samples difficult to test on many high throughput PCR machines.

The present invention solves the above problems by providing a method of collecting viral samples from the upper airway of a patient that is accurate, easily self-administered, and cost effective. Because the inventive sample collection method can be self-administered, it also eliminates the exposure of healthcare personnel.

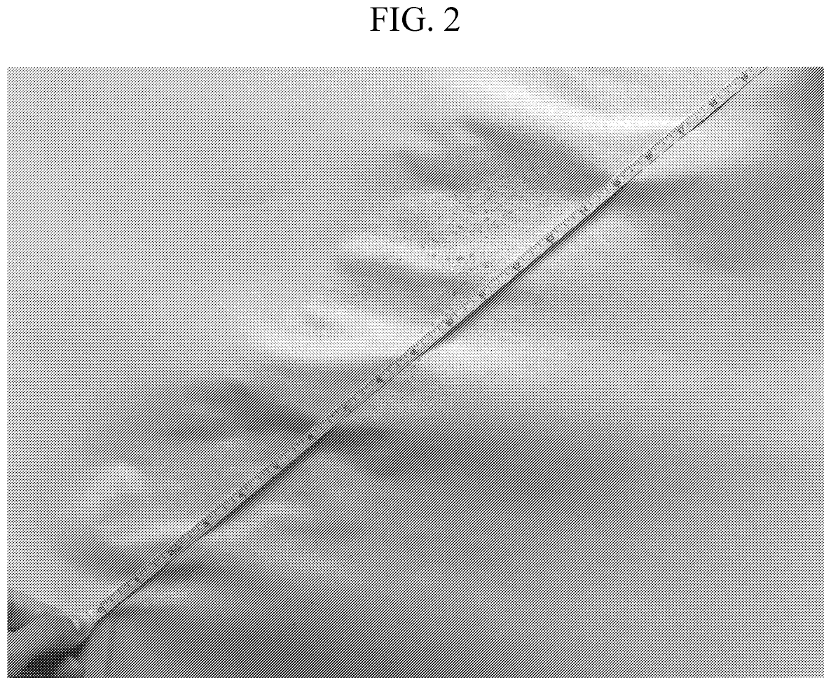

The self-administered collection method comprises a nasal lavage wherein the naval passages are washed using dyed saline sprayed through an atomizer. The lavaged saline will bathe the entirety of the nasal mucosa, including that of the olfactory groove, before being allowed to pass from the back of the nose into the back of the throat where the saline is then expectorated into a specimen collection container that has a stabilizing agent. From gastric to bronchoalveolar, lavage is a well-accepted specimen collection technique in medicine. The atomizer creates a large, but gentle, plume of saline that adequately bathes the surface of the entire nasal cavity, including the olfactory groove up high and all of the surfaces of the nasal turbinates (FIG. 2). This increases the chance of collecting viral particles, if present, and decreases the risk of a false negative result. Additionally, the dyed saline allows the patient to both taste and see that the fluid that is spit into the specimen collection container is truly the same fluid that lavaged the nasal passages.

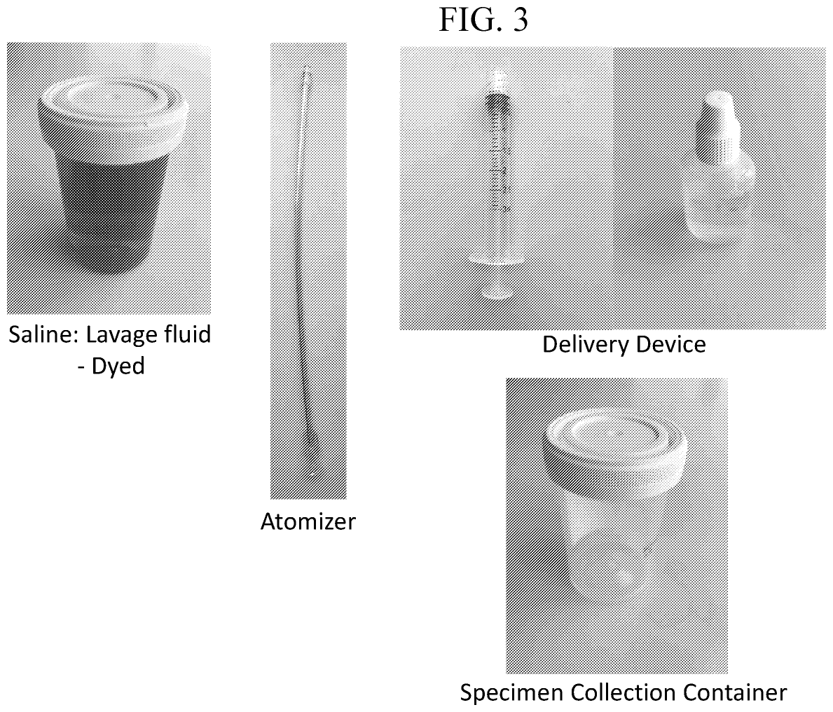

The present invention further relates to a kit for collection of a biological sample from a subject to be used for testing for a virus. The specimen collection kit includes a collection vehicle (i.e., the dyed saline as the lavage fluid), a 2-3 cc syringe or similarly sized squeeze bottle delivery device, atomizer that can attach to the delivery device, and a specimen collection container including a stabilizing reagent (FIG. 3).

Possible modifications to the collection method and collection kit will be studied. For example, it may be necessary for the atomizer to be short and/or to have a stopper on it to prevent the patient from inserting the atomizer too far up the nose. The specimen collection container could be modified to have a one way valve in order to prevent patients from drinking the stabilizing reagent. Modifications can also be made to make the sample collection process easier for self-administration or administration to children. One possible modification is that the lavaged saline could be blown from the nose as opposed to expectorating it from mouth. The expelled saline could then be swabbed for testing as it has bathed the entire nasal passage.

### List of Enumerated Embodiments

Embodiment 1. A method of obtaining a biological sample from the nose, nasal cavities, nasal pharynx, oral mucosa, or saliva of a subject for detection of a virus, the method comprising

(a) inserting into the nose of the subject a saline solution comprising a dye and

(b) collecting fluid from the nose or mouth of the subject.

Embodiment 2. The method of Embodiment 1, wherein, in step (a), the saline solution is allowed to flow from the nose to the mouth of the subject through the nasal passage and the nasal pharynx.
Embodiment 3. The method of Embodiment 1 or 2, wherein the saline solution is allowed to wash over the contents and walls of the nasal cavities, nasal pharynx, oral mucosa, or combination thereof.
Embodiment 4. The method of any one of Embodiments 1-3, wherein the saline solution is atomized.
Embodiment 5. The method of any one of Embodiments 1-4, further comprising (c) determining whether the fluid collected in step (b) contains a sufficient amount of the saline solution based on the color of the fluid.
Embodiment 6. The method of Embodiment 5, wherein if the fluid collected in step (b) is determined to not contain a sufficient amount of the saline solution, steps (a)-(c) are repeated.
Embodiment 7. The method of Embodiment 5, wherein if the fluid collected in step (b) is determined to contain a sufficient amount of the saline solution, the fluid collected in step (b) is determined to be an acceptable sample for subsequent use in detection of a virus.
Embodiment 8. The method of Embodiment 5, wherein step (c) further comprises determining whether the fluid collected in step (b) contains a sufficient amount of the saline solution based on the taste of the fluid.
Embodiment 9. The method of any one of Embodiments 1-8, wherein the dye is safe for ingestion.
Embodiment 10. The method of any one of Embodiments 1-9, wherein the method is self-administered.
Embodiment 11. The method of any one of Embodiments 1-10, wherein the subject is human.
Embodiment 12. The method of any one of Embodiments 1-11, wherein the virus is a coronavirus, a rhinovirus, an influenza virus, a parainfluenza virus, or a respiratory syncytial virus
Embodiment 13. The method of any one of Embodiments 1-12, wherein the virus is SARS-CoV-2.
Embodiment 14. A method of detecting a virus in a biological sample from a subject, the method comprising

(a) inserting into the nose of the subject a saline solution comprising a dye;

(b) collecting fluid from the nose or the mouth of the subject; and

(c) detecting presence of a virus in the fluid collected in step (b).

Embodiment 15. The method of Embodiment 14, wherein, in step (a), the saline solution is allowed to flow from the nose to the mouth of the subject through the nasal passage and the nasal pharynx.
Embodiment 16. The method of Embodiment 14 or 15, wherein the saline solution is allowed to wash over the contents and walls of the nasal cavities, the nasal pharynx, the oral mucosa, or a combination thereof.
Embodiment 17. The method of Embodiment 14, wherein step (c) is preceded by the step of determining whether the fluid collected in step (b) from the mouth of the subject contains a sufficient amount of the saline solution based on the color of the fluid.
Embodiment 18. The method of Embodiment 17, wherein if the fluid collected in step (b) is determined to contain an insufficient amount of the saline solution, steps (a) and (b) are repeated.
Embodiment 19. The method of Embodiment 17 or 18, wherein the step of determining whether the fluid collected in step (b) from the mouth of the subject contains a sufficient amount of the saline further comprises the step of determining whether the fluid collected in step (b) contains a sufficient amount of the saline solution based on the taste of the fluid.
Embodiment 20. The method of any one of Embodiments 14-19, wherein the saline solution is atomized.
Embodiment 21. The method of any one of Embodiments 14-20, wherein the dye is safe for ingestion.
Embodiment 22. The method of any one of Embodiments 14-21, wherein the presence of the virus is detected by polymerase chain reaction (PCR).
Embodiment 23. The method of any one of Embodiments 14-22, wherein the subject is human.
Embodiment 24. The method of any one of Embodiments 14-23, wherein the virus is a coronavirus, a rhinovirus, an influenza virus, a parainfluenza virus, or a respiratory syncytial virus.
Embodiment 25. The method of any one of Embodiments 14-24, wherein the virus is SARS-CoV-2.
Embodiment 26. A method of determining whether a subject is infected with a virus, the method comprising

(a) detecting the presence of a virus in a biological sample obtained from the subject, wherein the biological sample comprises fluid collected from the nose or the mouth of the subject after a saline solution comprising a dye is inserted into the nose of the subject and allowed to wash over the contents and walls of the nasal cavity, the nasal passage, the nasal pharynx, or combinations thereof.

Embodiment 27. The method of Embodiment 26, wherein the biological sample comprises fluid collected from the mouth of the subject after a saline solution comprising a dye is inserted into the nose of the subject and allowed to flow from the nose to the mouth of the subject through the nasal passage, the nasal pharynx, the oral mucosa, or a combination thereof.
Embodiment 28. The method of Embodiment 27, wherein the fluid collected from the mouth subject is determined to contain a sufficient amount of the saline solution based on the color of the fluid.
Embodiment 29. The method of Embodiments 26 or 27, wherein the fluid collected from the mouth subject is determined to contain a sufficient amount of the saline solution based on the taste of the fluid.
Embodiment 30. The method of any one of Embodiments 26-29, wherein the saline solution is atomized.
Embodiment 31. The method of any one of Embodiments 26-30, wherein the dye is safe for ingestion.
Embodiment 32. The method of any one of Embodiments 26-31, wherein the presence of the virus is detected by polymerase chain reaction (PCR).
Embodiment 33. The method of any one of Embodiments 26-32, wherein the subject is human.
Embodiment 34. The method of any one of Embodiments 26-33, wherein the virus is a coronavirus, a rhinovirus, an influenza virus, a parainfluenza virus, or a respiratory syncytial virus.
Embodiment 35. The method of any one of Embodiments 26-34, wherein the virus is SARS-CoV-2.
Embodiment 36. A kit for performing the method of any one of Embodiments 1-35, wherein the kit comprises a saline solution comprising a dye.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of obtaining a biological sample from the nose, nasal cavities, nasal pharynx, oral mucosa, or saliva of a subject for detection of a virus, the method comprising
(a) inserting into the nose of the subject a saline solution comprising a dye and
(b) collecting fluid from the nose or mouth of the subject.

2. The method of claim 1, wherein, in step (a), the saline solution is allowed to flow from the nose to the mouth of the subject through the nasal passage and the nasal pharynx.

3. The method of claim 1, wherein the saline solution is allowed to wash over the contents and walls of the nasal cavities, nasal pharynx, oral mucosa, or combination thereof.

4. The method of claim 1, wherein the saline solution is atomized.

5. The method of claim 1, further comprising (c) determining whether the fluid collected in step (b) contains a sufficient amount of the saline solution based on at least one of the color of the fluid and the taste of the fluid.

6. The method of claim 5, wherein:
if the fluid collected in step (b) is determined to not contain a sufficient amount of the saline solution, steps (a)-(c) are repeated; and
if the fluid collected in step (b) is determined to contain a sufficient amount of the saline solution, the fluid collected in step (b) is determined to be an acceptable sample for subsequent use in detection of a virus.

7-9. (canceled)

10. The method of claim 1, wherein the method is self-administered.

11. (canceled)

12. The method of claim 1, wherein the virus is a coronavirus, a rhinovirus, an influenza virus, a parainfluenza virus, or a respiratory syncytial virus

13. The method of claim 1, wherein the virus is SARS-CoV-2.

14. A method of detecting a virus in a biological sample from a subject, the method comprising
(a) inserting into the nose of the subject a saline solution comprising a dye;
(b) collecting fluid from the nose or the mouth of the subject; and
(c) detecting presence of a virus in the fluid collected in step (b).

15. The method of claim 14, wherein, in step (a), the saline solution is allowed to flow from the nose to the mouth of the subject through the nasal passage and the nasal pharynx.

16. The method of claim 14, wherein the saline solution is allowed to wash over the contents and walls of the nasal cavities, the nasal pharynx, the oral mucosa, or a combination thereof.

17. The method of claim 14, wherein step (c) is preceded by the step of determining whether the fluid collected in step (b) from the mouth of the subject contains a sufficient amount of the saline solution based on the color of the fluid.

18. The method of claim 17, wherein if the fluid collected in step (b) is determined to contain an insufficient amount of the saline solution, steps (a) and (b) are repeated.

19. The method of claim 17, wherein the step of determining whether the fluid collected in step (b) from the mouth of the subject contains a sufficient amount of the saline further comprises the step of determining whether the fluid collected in step (b) contains a sufficient amount of the saline solution based on the taste of the fluid.

20. The method of claim 14, wherein the saline solution is atomized.

21. (canceled)

22. The method of claim 14, wherein the presence of the virus is detected by polymerase chain reaction (PCR).

23. (canceled)

24. The method of claim 14, wherein the virus is a coronavirus, a rhinovirus, an influenza virus, a parainfluenza virus, or a respiratory syncytial virus.

25-35. (canceled)

36. A kit for performing the method of claim 1, wherein the kit comprises a saline solution comprising a dye.

37. The method of claim 14, further comprising determining whether the subject is infected with the virus based upon the detecting in step (c).
